(19) United States
(12) Patent Application Publication
(10) Pub. No.: US 2026/0273300 A1
(43) Pub. Date: Sep. 17, 2026

(54) Systems and Methods for Modulating Electrical Activity in Excitable Biological Tissue Using Induced Electric Fields Generated by Time-Varying Magnetic Fields

(71) Applicant: Quantum Nanostim LLC, Palm Harbor, FL (US)

(72) Inventor: Thomas Reilly, Palm Harbor, FL (US)

(21) Appl. No.: 19/673,093

(22) Filed: May 11, 2026

Related U.S. Application Data
(63) Continuation-in-part of application No. 17/818,993, filed on Aug. 11, 2022, now Pat. No. 12,629,536.
Application No. 17/818,993 is a continuation-in-part of application No. PCT/US21/17783, filed on Feb. 12, 2021.

(51) Int. Cl. A61N 2/02; A61N 2/00

(52) U.S. Cl. CPC A61N 2/02; A61N 2/006

(57) ABSTRACT

Systems and methods are provided for modulating electrical activity in excitable biological tissue using electric fields induced by time-varying magnetic fields. A first time-varying magnetic field induces a subthreshold electric field that alters tissue excitability without evoking an action potential, and a second time-varying magnetic field induces a depolarizing electric field sufficient to evoke activation. Interaction of the induced electric fields enables controlled modulation, including selective activation and reduced stimulation thresholds. Operation may be open-loop, closed-loop, or hybrid, wherein closed-loop control utilizes sensed physiological responses, including evoked responses, and/or signals representative of the magnetic field detected by one or more sensing elements. Systems include one or more coils configured to generate spatially and temporally varying magnetic fields. The disclosed techniques are applicable to neural, cardiac, and other excitable tissues, and enable stimulation without direct electrode-tissue contact.

### CROSS-REFERENCE TO RELATED APPLICATION

This application is a continuation-in-part of U.S. Pat. App. Ser. No 17/818,993, filed Aug. 11, 2022, titled Device for, and method of, neuromodulation with closed-loop micromagnetic hybrid waveforms to relieve pain, which in turn claims priority to PCT application PCT/US21/17783, filed on Feb. 12, 2021, titled Device for, and method of, neuromodulation with closed-loop micromagnetic hybrid waveforms to relieve pain, which in turn claims priority to U.S. patent application serial number 62/975,811, filed on Feb. 13, 2020, titled Device for, and method of, stimulation with closed loop hybrid waveform to relieve pain.

### FIELD

This invention relates to the field of electromagnetic stimulation of biological tissue and, more particularly, to methods and devices for modulating electrical activity in excitable biological tissue using induced electric fields generated by time-varying magnetic fields to modulate tissue excitability and to control spatial, temporal, and directional characteristics of the induced electric fields.

### BACKGROUND

Electrical stimulation systems often rely on direct electrode-tissue interfaces to deliver current to biological tissue. Such systems are limited by electrochemical interactions, impedance variability, electrode degradation, tissue damage, and reduced effectiveness in the presence of fibrotic encapsulation, glial scar, or other biological barriers. Over time, these limitations reduce the reliability of stimulation and constrain the ability to maintain consistent therapeutic outcomes.

Magnetic stimulation techniques have been explored as an alternative to electrode-based systems. Conventional magnetic stimulation systems, however, lack the ability to control induced electric fields at implantable scales for selective activation of specific tissue structures, directional targeting, and adaptive control based on physiological feedback.

Conventional magnetic stimulation approaches apply suprathreshold pulses to evoke action potentials in target tissue, requiring the induced electric field to exceed the activation threshold in a single step. The energy required to achieve suprathreshold stimulation in a single pulse is substantial because the activation threshold remains at its resting level throughout the stimulation. Existing systems lack mechanisms to precondition or prime excitable biological tissue before delivering an activation pulse. Without preconditioning, the full suprathreshold field intensity is delivered in each stimulation pulse, limiting precision and increasing energy consumption.

Additionally, conventional systems are limited in their ability to dynamically alter tissue excitability, achieve selective recruitment of specific neural or cardiac structures, maintain efficacy in the presence of impedance variability or encapsulation, and integrate multi-modal stimulation and sensing.

What is needed is a method and device for modulating electrical activity in excitable biological tissue that conditions the tissue using a subthreshold electric field to alter its excitability state, controls spatial, temporal, and directional characteristics of induced electric fields, operates without direct electrical contact, adapts stimulation in response to physiological feedback, and modulates tissue excitability through conditioning and priming mechanisms.

Restated, there remains a need for systems and methods capable of modulating electrical activity in excitable biological tissue using induced electric fields that alter an excitability state of the tissue prior to activation, control spatial, temporal, and directional characteristics of the induced electric field, operate without direct electrical contact with the tissue, and adapt stimulation based on feedback derived from physiologic responses and/or characteristics of the induced electric fields generated by one or more time-varying magnetic fields. Such systems may further incorporate mechanisms for estimating a functional state of the excitable biological tissue based on one or more sensed signals, and for adjusting one or more stimulation parameters based on the estimated state to achieve a desired physiologic response. In some implementations, feedback may be derived from one or more sensing modalities, including physiologic sensing and sensing of the induced electric fields generated by one or more time-varying magnetic fields.

### SUMMARY

The methods and devices described herein modulate electrical activity in excitable biological tissue using electric fields induced by time-varying magnetic fields. The approach uses a two-stage modulation process in which a subthreshold electric field first conditions the excitable biological tissue, and a depolarizing electric field then activates the conditioned tissue.

In the two-stage modulation process, a first time-varying magnetic field generates a subthreshold electric field within excitable biological tissue that alters an excitability state of the tissue without evoking an action potential. A second time-varying magnetic field generates a depolarizing electric field within the excitable biological tissue sufficient to evoke activation. The subthreshold electric field of the first time-varying magnetic field establishes a subthreshold excitability state that lowers a depolarization threshold, thereby altering the response of the tissue to the depolarizing electric field of the second time-varying magnetic field.

The induced electric field interacts with cellular membranes, influencing transmembrane potentials, ion channel dynamics, excitability thresholds, and electrical signaling behavior. Through this interaction the subthreshold conditioning modifies ion channel states, shifts depolarization thresholds, alters membrane responsiveness, and alters tissue electrical properties including impedance or conductivity. These changes enhance the effect of the subsequent depolarizing electric field, enabling enhanced recruitment efficiency, selective activation of tissue structures, reduced stimulation thresholds, and improved energy efficiency. The induced electric field is generated by magnetic fields alone or in combination with one or more electrical stimulation signals.

Timing of the first and second fields depends on the intended therapeutic effect. The two time-varying magnetic fields may be delivered either simultaneously or sequentially. In simultaneous delivery, both fields are applied at the same time and may differ in parameters such as frequency, amplitude, or phase to create constructive or modulatory interactions. In sequential delivery, the first field conditions the tissue, followed by a defined temporal delay, after which the second field delivers the depolarizing stimulus. The induced electric fields may share or differ in key parameters, including frequency, amplitude, phase polarity, relative phase, or harmonic content, depending on the desired neuromodulatory outcome.

In certain embodiments, sensing is performed using one or more sensing elements configured to detect physiologic responses, biomarkers, and/or signals representative of a magnetic field. Sensed signals may be used to estimate a state of the excitable biological tissue and to control delivery of one or more time-varying magnetic fields and corresponding induced electric fields.

In some embodiments, one or more sensing coils may be configured to detect signals representative of a delivered time-varying magnetic field, a corresponding induced electric field, a stimulation artifact, decay characteristics thereof, or combinations thereof. Information derived from the sensed signals may be used alone or in combination with one or more evoked physiologic responses and/or biomarkers, including evoked compound action potentials (ECAPs), electrophysiological signals, activation patterns, or combinations thereof, to determine, adjust, or optimize one or more sensing parameters, including a blanking interval, sensing recovery interval, detection window, filtering characteristic, artifact suppression parameter, or combinations thereof.

In some embodiments, estimation of stimulation artifact decay characteristics, induced electric field decay characteristics, related temporal response characteristics, and/or one or more evoked physiologic responses and/or biomarkers may facilitate reduction or adjustment of a blanking interval, sensing recovery interval, or other post-stimulation sensing window relative to conventional stimulation systems. In some embodiments, one or more sensing parameters may be dynamically adjusted based on the estimated decay characteristics, detected physiologic responses, detected biomarkers, or combinations thereof.

Control of one or more time-varying magnetic fields and corresponding induced electric fields may include feedback control, feedforward control, predictive control, adaptive control, or combinations thereof. In some embodiments, one or more stimulation parameters may be adjusted based on sensed physiologic responses, evoked physiologic responses, biomarkers, impedance characteristics, detected motion, posture, activity state, sleep-wake state, circadian characteristics, time of day, historical response patterns, predicted physiologic changes, or combinations thereof.

In some embodiments, feedforward control may be used to modify one or more stimulation parameters in anticipation of expected or predicted changes in physiologic state, impedance characteristics, motion, posture, activity level, sleep-wake cycle, time of day, evoked physiologic responses, biomarkers, or combinations thereof. Motion, posture, or activity state may be detected using one or more accelerometers, inertial measurement units, gyroscopes, pressure sensors, position sensors, wearable sensors, implantable sensors, external sensors, or combinations thereof.

In some embodiments, impedance characteristics may include tissue impedance, electrode-tissue interface impedance, magnetic coupling characteristics, conductivity characteristics, impedance trends over time, spatial impedance distributions, temporal impedance variations, or combinations thereof. One or more stimulation parameters may be adjusted based on detected or estimated impedance characteristics to influence one or more induced electric fields, physiologic responses, stimulation thresholds, recruitment characteristics, conduction characteristics, synchrony characteristics, or combinations thereof.

The process includes feedback, or data from sensors used to adjust the generation of the signals, which depends upon the methodology of operation. The two-stage modulation process can operate in an open-loop configuration, a closed-loop configuration, or a hybrid configuration combining both approaches.

In one embodiment, modulation is performed in an open-loop configuration without sensing or adjusting based on a physiological response from the excitable biological tissue. The parameters of the first and second time-varying magnetic fields are predetermined and selected based on anatomical targeting, patient-specific calibration, or therapeutic objectives.

In another embodiment, modulation is performed in a closed-loop configuration. The method includes sensing an evoked response from the excitable biological tissue and adjusting one or more parameters of at least one of the induced electric fields based on the sensed evoked response. The generating, sensing, and adjusting steps are repeated iteratively to modulate electrical activity of the excitable biological tissue. One or more parameters of the induced electric field are adjusted to maintain an evoked response characteristic within a predefined range based on one or more response characteristics associated with excitability or activation of the excitable biological tissue.

In certain embodiments, the predefined range comprises a target operating window including one or more setpoints, upper bounds, lower bounds, or allowable variability thresholds associated with a stimulation dose, evoked response, or inferred activation state. The controller is configured to maintain the evoked response, stimulation dose, or volume of tissue activation within the target operating window by adjusting one or more stimulation parameters in response to sensed physiologic signals. In some embodiments, the controller reduces variability in the evoked response or inferred activation state over time, including during changes in posture, tissue position, lead position, or spacing between the field-generating element and the excitable biological tissue.

In another embodiment, modulation is performed in a hybrid configuration that combines open-loop and closed-loop approaches. In the hybrid configuration, open-loop stimulation is supplemented by periodic or conditional feedback-based adjustment.

In certain embodiments, neural activation is governed by temporal characteristics of the applied time-varying magnetic field on a sub-millisecond timescale, including waveform shape, phase duration, interphase interval, and rise and fall dynamics, rather than solely by stimulation repetition rate.

In some embodiments, one or more time-varying magnetic fields comprise stimulation waveforms having monophasic, biphasic, multiphasic, burst, continuous, discontinuous, periodic, non-periodic, deterministic, stochastic, arbitrary, Gaussian, sinusoidal, sinusoid-derived, rectangular, square, triangular, ramped, trapezoidal, rectified, asymmetric, unbalanced, or other waveform profiles, wherein waveform characteristics including amplitude, phase, temporal profile, pulse width, frequency, frequency content, duty cycle, spectral composition, phase relationships, rise characteristics, fall characteristics, burst characteristics, or combinations thereof may be selected to influence one or more characteristics of one or more induced electric fields and/or one or more corresponding physiologic responses.

In certain embodiments, one or more characteristics of the induced electric field within excitable biological tissue are determined, at least in part, by a time-varying characteristic of the magnetic field, including one or more temporal derivatives thereof, such that one or more spectral components of the waveform may differentially influence neural activation, excitability, recruitment characteristics, spatial distribution of activation, and/or corresponding physiologic responses.

In certain embodiments, waveform characteristics, including temporal profile and frequency content, influence the induced electric field and corresponding physiologic response. Variation in waveform shape may result in differences in the time-varying magnetic field and corresponding induced electric field, thereby affecting activation magnitude, spatial distribution, or selectivity of tissue response.

Accordingly, stimulation delivered at relatively low repetition rates may nevertheless produce effective neural activation and evoked responses, including ECAPs, due to intra-waveform temporal structure containing higher-frequency spectral components.

In certain embodiments, stimulation repetition rate is decoupled from the temporal dynamics responsible for neural activation, such that stimulation events may occur at relatively low frequencies while intra-event waveform characteristics govern the magnitude, spatial distribution, orientation, and selectivity of induced electric fields.

In the closed-loop and hybrid configurations, sensing is performed using one or more sensing elements configured to detect physiologic responses, including evoked responses, from the excitable biological tissue. Sensed signals include: evoked compound action potentials (ECAPs); late responses including F-waves, H-reflexes, and A-waves; evoked synaptic activity potentials (eSAPs); Dorsal Root Potentials (DRP); electromyographic (EMG) signals; electrocardiographic signals; intracardiac electrograms; electrograms; and other electrophysiological responses. Sensed parameters can further include conduction velocity, rheobase, and chronaxie. Feedback based on sensed signals maintains a target response, dynamically adjusts stimulation parameters, and optimizes energy delivery.

In certain embodiments, sensing is further performed using one or more sensing elements configured to detect a signal representative of a magnetic field generated by one or more coils. In certain embodiments, the signal representative of the magnetic field comprises an induced voltage corresponding to an electromotive force (EMF) generated within a sensing coil in response to the time-varying magnetic field, although other magnetic field sensing modalities may be used.

In certain embodiments, one or more sensed signals, including physiologic responses and/or signals representative of a magnetic field, are processed to estimate a state of the excitable biological tissue. The estimated state may include one or more characteristics such as excitability, activation threshold, recruitment level, conduction properties, spatial distribution of activation, or other physiologic or biophysical parameters. The estimated state may be used to control one or more characteristics of a time-varying magnetic field and a corresponding induced electric field, including adjusting one or more stimulation parameters to achieve a desired response, and may be determined using one or more models, algorithms, or signal processing techniques.

Conventional electrode-based stimulation systems produce a stimulus artifact that requires a blanking interval, which is a period during or after delivery of a stimulation pulse in which sensing is suppressed to avoid saturating the sensing electronics. Because the induced electric fields described herein are generated by magnetic induction rather than direct electrical contact with tissue, the resulting stimulus artifact may be reduced, modified, redistributed, or otherwise differ in amplitude, temporal profile, or spatial characteristics relative to electrode-based systems. In some embodiments, these differences permit a reduction in the duration of the blanking interval and may enable detection of evoked responses during or immediately following stimulation, including within an early post-stimulation time window in which the stimulus artifact does not fully obscure physiologic signals. This earlier detection window may enable characterization of latency and early response components for real-time adjustment of one or more stimulation parameters, which may be more difficult or less reliable in systems requiring longer blanking intervals.

In certain embodiments the system is configured to detect evoked neural responses propagating in one or more directions, including orthodromic propagation, antidromic propagation, or combinations thereof. Directional detection is achieved using one or more sensing elements positioned proximal, distal, rostral, caudal, ipsilateral, contralateral, or otherwise spatially offset relative to a stimulation site, such that directionally propagating evoked responses are distinguishable based on one or more signal characteristics including latency, waveform morphology, temporal sequence, amplitude, or combinations thereof.

In some embodiments, detection of directionally propagating evoked responses is facilitated by properties of micromagnetic stimulation, including reduced or altered stimulation artifact, directional field generation, spatial selectivity, or improved temporal separation between stimulation artifact and neural response, thereby enabling detection of early response components and directional propagation characteristics that may be obscured in conventional electrode-based stimulation systems.

In certain implementations, orthodromic and antidromic responses are analyzed independently or comparatively to characterize neural pathway engagement, conduction properties, recruitment dynamics, or functional state of the excitable biological tissue. Differences between orthodromic and antidromic responses, including variations in latency, amplitude, waveform morphology, or temporal dispersion, are used to infer directional activation patterns and to adjust one or more stimulation parameters, including to adjust one or more stimulation parameters, including amplitude, frequency, pulse width, waveform shape, temporal characteristics, spatial distribution of induced electric fields, or combinations thereof.

Closed-loop control further includes estimation of a neural state based on one or more characteristics of sensed physiologic signals, including signal presence, reduction, saturation, latency, waveform morphology, recruitment behavior, temporal patterns, or combinations thereof. The estimated neural state corresponds to states associated with increased or reduced neural activity, reflecting differences in neural recruitment, propagation, synchrony, or other functional properties of excitable biological tissue. Changes in evoked responses, including variations in amplitude, reduction, saturation, or absence of signals such as ECAPs, occur under conditions consistent with changes in neural activity, including reduced propagation, altered synchrony, refractory effects, or inhibitory phenomena. The system adjusts stimulation parameters based on the sensed physiologic signals to navigate between functional states of neural activity.

Neural state estimation is the process of inferring the current functional condition of excitable biological tissue, such as whether the tissue exhibits increased or reduced neural activity, based on characteristics of sensed physiologic signals rather than direct measurement of the tissue state itself.

In the system, neural state estimation does not require detection of an evoked response following every stimulation pulse. Instead, the neural state is inferred from trends or changes in signal characteristics observed across detected responses, such as shifts in amplitude, latency, or recruitment behavior over time. The sensing system operates continuously, intermittently, or periodically depending on the application.

A benefit of the system includes that evoked responses remain detectable at higher stimulation frequencies, including frequencies at which such responses are diminished or not observable using conventional stimulation approaches. This detectability enables characterization of neural activity, monitoring of recruitment, and adjustment of stimulation parameters under conditions where conventional systems do not provide reliable feedback. Such detectability is associated with properties of the induced electric field, including spatial distribution, temporal characteristics, or reduced interference between stimulation and sensing.

In some embodiments, such detectability is associated with intra-waveform temporal dynamics that contain higher-frequency spectral components independent of the stimulation repetition rate.

Stimulation is delivered at a frequency that permits temporal separation of evoked responses, enabling analysis of individual response characteristics including onset latency, peak latency, recovery behavior, and trial-to-trial reproducibility. Temporal separation enables characterization of response stability, variability, and recovery dynamics, which are used to assess neural responsiveness and optimize stimulation parameters. Signal characteristics include measures of variability, noise, or signal-to-noise ratio, which are used to assess signal quality and stability of neural activation.

In certain embodiments, temporal separation of stimulation events is achieved while intra-waveform temporal characteristics independently govern neural activation, enabling evoked responses to be generated at repetition rates lower than those conventionally used in electrical stimulation systems.

Modulation may also be achieved using hybrid combinations of magnetic and electrical stimulation. Hybrid configurations include magnetic stimulation followed by electrical stimulation, electrical stimulation followed by magnetic stimulation, simultaneous delivery of both modalities, and coordinated temporal modulation between them. Hybrid stimulation enhances selectivity, reduces energy requirements, improves recruitment efficiency, and enables additional control dimensions. One or more of the first or second fields are generated by electrical stimulation, magnetic stimulation, or combinations thereof. Electrical and magnetically induced signals are combined to form hybrid evoked activity potentials (HEAPs), which provide additional sensing information for feedback-based control.

The time-varying magnetic field is generated using waveforms including sinusoidal, rectified sinusoidal, Gaussian, triangular, rectangular, square, pulsed, burst, amplitude-modulated, frequency-modulated, phase-modulated, or arbitrary waveforms, as well as harmonically enriched signals and waveforms having harmonic content, non-linear profiles, or composite multi-component configurations. Such waveforms are balanced or unbalanced. A single waveform or multiple waveforms are configured to produce one or more functional components, including subthreshold modulation, depolarizing activation, or combinations thereof.

Waveform characteristics are selected to control one or more properties of the induced electric field within tissue, including spatial distribution, temporal dynamics, directional orientation, magnitude, and gradient. Additionally, waveform characteristics are selected to modulate one or more physiological responses, including excitability, conduction, synchronization, or propagation of electrical activity, and are configured to alter electrical properties of tissue including impedance or conductivity, thereby modifying responsiveness to subsequent stimulation.

The waveform has asymmetric characteristics in some configurations, including unequal positive and negative phases or unequal temporal or amplitude characteristics. Waveforms are delivered as single signals, sequential signals, overlapping signals, or temporally offset signals, including configurations in which a first signal alters tissue responsiveness and a second signal produces activation. Parameters of the waveform include frequency, amplitude, pulse width, phase, phase polarity, relative phase, duty cycle, harmonic composition, and temporal offset.

In certain embodiments, spatial distribution of induced electric fields is controlled, shaped, or modulated through coordinated modulation of multiple stimulation parameters across two or more coils or inductive elements, including amplitude ratio, phase offset, polarity, temporal delay, waveform shape, and coil orientation, individually or in combination, such that corresponding induced electric fields interact through superposition, including constructive and destructive interference, to steer, focus, broaden, or dynamically translate regions of neural activation within excitable biological tissue.

The spatial distribution of the induced electric field is controlled through techniques including coil orientation control, multi-coil interference patterns, phase-controlled field superposition, and geometric positioning relative to tissue. The induced electric field is generated by one or more coils, including planar coils, cylindrical coils, microcoils (µcoils), or inductors. One or more planar coils are configured to tilt about one or more axes to steer induced electric fields, and one or more cylindrical coils are configured to tilt about one or more axes or to rotate about an axis over a range up to 360 degrees to steer induced electric fields. Steering comprises controlling spatial orientation of the induced electric fields relative to the excitable biological tissue.

During operation, induced electric field vectors are aligned relative to the structural orientation of the excitable biological tissue, including neural pathways, axonal direction, or myocardial fiber orientation. Alignment of induced electric field vectors with structural orientation enables selective activation of specific tissue populations, reduction of off-target effects, and directional control of activation.

The induced electric field comprises spatial gradients that vary across excitable biological tissue, producing differential polarization along cellular structures including neural fibers or cellular networks. In certain embodiments, selective activation is further achieved by aligning induced electric field vectors with cellular or microstructural orientation within the tissue, including dendritic, axonal, or fiber-level organization. Such alignment enables preferential modulation of specific cell populations or circuits based on spatial orientation of their structural components. Spatial variation in the induced electric field results in selective activation, modulation, or inhibition of different regions or pathways. Spatial gradients are controlled by adjusting coil geometry, coil orientation, relative positioning of multiple coils, phase relationships, or waveform characteristics, and gradient-based field shaping differentially influences activation thresholds across spatially distinct regions.

Two or more time-varying magnetic fields are generated such that their corresponding induced electric fields interact within the excitable biological tissue. Such interaction includes constructive interference, resulting in increased effective field strength within a target region, as well as destructive interference, resulting in reduced or canceled fields within a non-target region. Interference is controlled by adjusting relative phase, timing, amplitude, spatial positioning, or orientation of the fields, and is used to enhance activation within a first region while reducing activation within a second region.

The resulting induced electric field produces activation within excitable biological tissue that includes central neural structures, peripheral neural structures, or combinations thereof. Peripheral activation includes activation of sensory fibers, motor fibers, autonomic fibers, or cutaneous receptors, contributing to observed physiologic or perceptual responses. The induced electric field is configured, through control of spatial distribution, orientation, or gradients, to preferentially modulate selected regions while reducing off-target activation.

Changes in neural activity are associated with spatial variations in the induced electric field, including field-gradient effects that produce differential membrane polarization along cellular structures. These effects are associated with mechanisms consistent with changes in membrane polarization, ion channel activity, neural synchrony, or combinations thereof, without requiring a specific underlying biophysical mechanism. Modulation of excitable biological tissue is achieved through controlled spatial distribution of induced electric fields, including through field gradients, directional alignment, and interaction between multiple fields, enabling selective modulation of target regions while reducing activation of non-target structures without requiring direct electrical contact with the tissue.

Because stimulation is delivered without direct electrical contact with the tissue, no electrode-tissue interface is required. The absence of a direct electrode-tissue interface provides reduced electrochemical effects at the tissue surface, improved performance in the presence of fibrotic encapsulation or other biological barriers, and reduced risk of tissue damage. The induced electric field is generated across intervening media, including blood, connective tissue, or fibrotic regions, enabling stimulation of tissue regions not accessible by electrodes.

In certain embodiments, micromagnetic stimulation produces an increase in a target physiologic response relative to electrical stimulation, including increases in magnitude, consistency, or efficiency of activation.

In some embodiments, the increase comprises at least about 25%, 50%, 100%, or more, including about 2×–10× relative to a comparator, depending on tissue, geometry, and waveform parameters.

In certain embodiments, the system achieves a desired physiologic response at reduced input current or power relative to electrical stimulation, thereby improving energy efficiency.

In some embodiments, one or more coils operate reliably when exposed to physiologic media, including blood and cerebrospinal fluid, while maintaining functional performance over time.

In certain embodiments, coils are positioned relative to target tissue, including nerve roots, with orientations selected to control induced electric field directionality and spatial distribution.

In one non-limiting example, stimulation of a sacral nerve root produced an average rectal pressure of approximately 19.9 mmHg with micromagnetic stimulation compared to approximately 4.9 mmHg with electrical stimulation, and an average EMG power of approximately 8.7 mV² compared to approximately 1.2 mV², demonstrating a substantially increased response.

In a further non-limiting example, early human nerve conduction studies demonstrated that micromagnetic stimulation produced increased evoked response amplitudes relative to conventional electrical stimulation.

In certain implementations, micromagnetic stimulation may achieve increased response amplitudes while operating at reduced input current levels, including improvements on the order of at least about 10%, 25%, 50%, or greater, and reductions in current including at least about 2×, 3×, 4×, or more, depending on tissue type, stimulation parameters, and device configuration, thereby suggesting improved stimulation efficiency and selectivity.

In one non-limiting instance, an approximately 50% greater response amplitude was observed at approximately fourfold lower current.

The foregoing examples are illustrative and non-limiting, and greater or lesser improvements in response amplitude, efficiency, or selectivity may be achieved across different tissues, stimulation parameters, and device configurations.

In some embodiments, increased response amplitude at reduced current corresponds to improved energy efficiency, reduced power consumption, enhanced spatial selectivity of neural activation, or combinations thereof.

The foregoing example is illustrative and non-limiting, and similar or greater improvements may be achieved across different tissues, stimulation parameters, and device configurations.

The performance, efficiency, and durability characteristics described herein may be achieved independently or in combination with any of the stimulation, sensing, or control configurations described in this specification.

In certain embodiments, a first coil is configured to deliver a time-varying magnetic field to induce an electric field in excitable biological tissue, and one or more additional coils, positioned proximal, distal, or otherwise spatially offset relative to the first coil, are configured to operate in a passive sensing mode to detect a signal representative of magnetic flux or the time-varying magnetic field generated by the first coil.

The detected signal may be used to estimate one or more characteristics of the delivered magnetic field or a corresponding induced electric field, including amplitude, phase, temporal profile, frequency content, spatial distribution, or waveform fidelity, and to adjust one or more stimulation parameters of the first coil, including amplitude, phase, pulse width, frequency, waveform shape, duty cycle, coil selection, or timing, to maintain a desired stimulation field or physiologic response.

In some embodiments, sensing of the magnetic field may be performed using one or more coils or other magnetic field sensing elements, including electromagnetic, magnetoresistive, Hall-effect, fluxgate, or other magnetic field sensors configured to detect a signal representative of the time-varying magnetic field.

In certain embodiments, field-based sensing and physiologic sensing are used in combination to adjust one or more stimulation parameters. For example, a signal representative of a magnetic field and one or more physiologic responses may be jointly processed to estimate a state of the excitable biological tissue and to control delivery of the time-varying magnetic field. In some implementations, one sensing modality may be used to calibrate, validate, or supplement the other, or to provide feedback under conditions in which the other sensing modality is unavailable or less reliable.

In certain embodiments, estimation, processing, or control of the detected signal may be performed using one or more mathematical, computational, algorithmic, heuristic, model-based, data-driven, or machine learning approaches, or combinations thereof, and may be implemented in hardware, software, firmware, or any combination thereof, and is not limited to any specific method of analysis, representation, or implementation.

The methods and devices described herein are applicable to any excitable biological tissue, including neural tissue, cardiac tissue, smooth muscle tissue, skeletal muscle tissue, autonomic and enteric nervous system tissue, sensory receptor cells, neuroendocrine cells, pacemaker cells, glial cells, vascular-associated cells, and other electrically responsive cellular networks. Modulation of electrical activity includes altering excitability, conduction, synchronization, or propagation of electrical signals within or across such tissues. Subthreshold electric fields alter excitability and conduction properties of the tissue, including impedance, conductivity, or effective field penetration, thereby influencing electrical propagation and responsiveness to subsequent stimulation. Modulation of electrical activity also occurs independently of synaptic mechanisms.

Where the excitable biological tissue comprises neural tissue, target tissue includes central nervous system structures, peripheral nerves, ganglionic structures, and spinal cord regions including the dorsal horn. Modulation includes inhibition of nociceptive transmission, alteration of synaptic activity, or modulation of neural signaling, with or without paresthesia. Beyond pain modulation, modulation of neural tissue includes a range of functional effects including alteration of sensory, motor, autonomic, or cognitive signaling.

Modulation of neural tissue further includes desynchronization of pathological neural activity, disruption of aberrant circuits, restoration of functional signaling, and network-level reorganization. These effects result from one or more mechanisms acting individually or in combination.

The methods and devices described herein also apply to cardiac tissue, including myocardium and components of the cardiac conduction system. The methods and devices enable modulation of cardiac electrical activity using electric fields induced by time-varying magnetic fields without direct electrode-tissue contact. Unlike conventional cardiac stimulation systems that rely on direct current injection through electrodes, the methods and devices described herein enable non-contact, inductively coupled modulation of cardiac tissue, reducing or eliminating limitations associated with electrode-tissue interfaces including impedance variability, fibrosis, lead positioning constraints, and electrochemical effects.

The induced electric field is configured to modulate cardiac electrical activity through one or more mechanisms, including pacing or entrainment of cardiac rhythm, modulation of intrinsic pacemaking activity, suppression, termination, or prevention of arrhythmias, stabilization or alteration of conduction pathways, and modulation of conduction velocity or refractoriness. Modulation includes subthreshold modulation of myocardial excitability, enabling alteration of cardiac electrical behavior without continuous depolarization.

The induced electric field is generated across intervening media including blood, connective tissue, or fibrotic regions adjacent to cardiac tissue, enabling stimulation of cardiac tissue regions not accessible by electrodes, reducing dependence on precise electrode positioning, and improving performance in remodeled or fibrotic cardiac tissue. The stimulation source is positioned external to the myocardium, within vascular structures, or adjacent to cardiac tissue.

Due to anisotropic conduction properties of cardiac tissue, alignment of induced electric field vectors with myocardial fibers preferentially influences conduction pathways, enhances activation efficiency, and enables selective modulation of electrical propagation. Multiple coils or field-generating elements are configured to dynamically steer induced electric field vectors, generate spatially controlled electric field gradients, and selectively target regions of cardiac tissue.

Cardiac modulation is achieved using coordinated magnetic fields, including a first time-varying magnetic field configured to induce a subthreshold electric field that alters cardiac excitability and a second time-varying magnetic field configured to induce a depolarizing electric field sufficient to evoke activation or modify conduction. The interaction between these fields enables reduced stimulation thresholds, selective recruitment of cardiac tissue, and enhanced control of electrical propagation.

The system includes sensing capabilities configured to detect cardiac electrical activity, including intracardiac electrograms, surface electrocardiograms, or other electrophysiological signals. Stimulation parameters are adjusted based on sensed cardiac activity to maintain cardiac rhythm within a target range, suppress or prevent arrhythmogenic events, optimize conduction characteristics, and improve hemodynamic performance. Control is based on heart rate variability, conduction intervals, timing relationships within the cardiac cycle, or detection of abnormal rhythms.

The time-varying magnetic field is delivered in synchrony with the cardiac cycle. Stimulation is gated relative to intrinsic cardiac activity to enhance modulation efficacy, reduce unintended activation, and improve safety and energy efficiency.

Similar principles of excitability modulation are applied to other excitable biological tissues, including smooth muscle such as gastrointestinal, vascular, respiratory, or urogenital muscle, as well as skeletal muscle and electrically responsive cellular networks including pacemaker cells and supporting cells, without requiring modification of the underlying stimulation architecture.

Modulation of electrical activity involves alterations in cellular excitability and synaptic efficacy. The induced electric fields influence membrane dynamics, ion channel behavior, and network-level signaling within the tissue. The subthreshold electric field alters an excitability state of the tissue without evoking an action potential, and such alteration includes changes in membrane polarization, ion channel activation thresholds, or synaptic responsiveness. These changes persist beyond the duration of the applied field and influence subsequent responses to stimulation.

Modulation involves synaptic plasticity mechanisms within neural tissue. Synaptic plasticity refers to processes by which synaptic strength is modified, including long-term potentiation (LTP) and long-term depression (LTD). The induced electric fields modulate synaptic efficacy by influencing pre-synaptic and post-synaptic signaling pathways, including mechanisms associated with neurotransmitter release, receptor activation, and intracellular signaling cascades. The modulation of electrical activity results in enhancement of synaptic transmission, suppression of synaptic transmission, or reorganization of neural network activity.

The subthreshold electric field functions as a priming stimulus that induces a metaplastic state within neural tissue. Metaplasticity refers to changes in neuronal or synaptic state that are induced by prior activity and that subsequently influence the ability of synapses to undergo plasticity. Such changes persist for extended durations, including minutes, hours, or longer, and decay over time. The first time-varying magnetic field induces a primed state that modifies the response of the tissue to subsequent stimulation, and the second time-varying magnetic field induces activation or further modulation that is influenced by the primed state. This interaction enables controlled modulation of neural activity through sequential or coordinated stimulation.

Modulation includes homosynaptic or heterosynaptic effects. Homosynaptic effects occur when changes in synaptic efficacy are confined to synapses that are directly influenced by stimulation, while heterosynaptic effects occur when modulation extends to neighboring or functionally connected synapses that are not directly activated. Heterosynaptic modulation involves interactions across dendritic compartments, intracellular signaling pathways, or intercellular communication within neural networks, enabling broader network-level modulation beyond directly stimulated regions.

Modulation effects persist after cessation of stimulation. The persistence of such effects is associated with plasticity-related processes, including the involvement of plasticity-related proteins or other intracellular mechanisms that stabilize changes in synaptic efficacy. Transient changes in excitability evolve into longer-lasting modifications, and decaying plasticity states are reinforced or prolonged through subsequent stimulation.

Modulation occurs through one or more mechanisms, including membrane-level effects, ion channel modulation, synaptic plasticity, metaplasticity, network-level interactions, or combinations thereof. The methods and devices described herein are not limited to any particular biological mechanism.

A device for modulating electrical activity in excitable biological tissue includes one or more coils configured to generate one or more time-varying magnetic fields and a control system configured to drive the one or more coils to produce a first time-varying magnetic field and a second time-varying magnetic field. The first and second time-varying magnetic fields are configured to induce respective electric fields that modulate excitability of the excitable biological tissue. The device further includes a sensing system configured to detect one or more physiologic responses from the excitable biological tissue, and the control system is configured to adjust one or more parameters of the induced electric fields based on the detected responses.

Devices include implantable or external systems with one or more coils, leads or flexible substrates, control electronics, and optional sensing elements. Coil types include planar coils, cylindrical coils, and microfabricated inductors. Structural features include expandable or conformable elements, shape-memory materials, and rotatable or tiltable assemblies. The device is configured to perform any of the methods described herein.

Optionally, devices are configured to operate within magnetic resonance imaging (MRI) environments, minimize radio frequency (RF) induced heating, and reduce induced currents from gradient fields. Materials and geometries are selected to enhance MRI compatibility.

Energy delivery is optimized through dual-field modulation and reduced stimulation thresholds. Because the subthreshold electric field lowers the depolarization threshold prior to delivery of the depolarizing electric field, the energy required to evoke activation is reduced compared to a single suprathreshold pulse. Optional features include energy harvesting components, nanogenerators, and inductive coupling for power delivery.

In certain embodiments, systems and methods described herein utilize one or more time-varying magnetic fields to induce electric fields in excitable biological tissue, wherein stimulation is controlled based on one or more of temporal characteristics, spatial distribution, waveform properties, or physiologic responses.

In certain embodiments, modulation of electrical activity includes subthreshold conditioning, depolarizing activation, or combinations thereof, delivered simultaneously, sequentially, or as part of a composite waveform.

In certain embodiments, one or more parameters of the induced electric field are controlled or adjusted, including amplitude, frequency, phase, waveform shape, temporal characteristics, spatial distribution, or combinations thereof, to influence electrical activity of the excitable biological tissue.

In certain embodiments, control of electrical activity includes closed-loop, open-loop, or hybrid configurations, including adjustment based on sensed physiologic responses, sensed field characteristics, or inferred neural state.

### BRIEF DESCRIPTION OF THE DRAWINGS

The invention can be best understood by those having ordinary skill in the art by reference to the following detailed description when considered in conjunction with the accompanying drawings in which:

FIG. 1A illustrates a two-stage modulation process with sequential delivery of subthreshold conditioning and depolarizing activation.

FIG. 1B illustrates a two-stage modulation process with simultaneous delivery of subthreshold conditioning and depolarizing activation.

FIG. 2 illustrates a closed-loop system configured to generate one or more time-varying magnetic fields that induce one or more electric fields within excitable biological tissue, detect one or more physiologic responses and/or signals representative of the magnetic field, and iteratively adjust one or more stimulation parameters to modulate electrical activity of the excitable biological tissue.

FIG. 3 illustrates a coil assembly configured to generate one or more time-varying magnetic fields and a sensing element configured to detect a signal representative of the magnetic field using a sensing coil.

FIG. 4 illustrates coil variations configured to tilt and/or rotate about one or more axes to steer one or more induced electric field vectors relative to excitable biological tissue.

### DETAILED DESCRIPTION

Reference will now be made in detail to the presently preferred embodiments, examples of which are illustrated in the accompanying drawings. Throughout the following detailed description, the same reference numerals refer to the same elements in all figures.

“Excitable biological tissue” includes any tissue, cellular network, biologic structure, or biologic system capable of generating, propagating, responding to, modulating, or being influenced by electrical activity, electromagnetic activity, electrochemical signaling, ionic signaling, membrane polarization, depolarization, hyperpolarization, action potentials, graded potentials, field effects, or other bioelectrical or biophysical signaling mechanisms, including without limitation neural tissue, cardiac tissue, smooth muscle tissue, skeletal muscle tissue, autonomic nervous system tissue, enteric nervous system tissue, sensory receptor cells, neuroendocrine cells, pacemaker cells, glial cells, vascular-associated cells, immune-associated cells, and combinations thereof.

“Time-varying magnetic field” refers to any magnetic field that varies in magnitude, direction, phase, spatial distribution, frequency, frequency content, or combinations thereof over time, and that is capable of generating, inducing, influencing, or modulating one or more electric fields within or relative to excitable biological tissue via electromagnetic induction or related electromagnetic interactions.

“Subthreshold electric field” refers to an induced electric field that alters, biases, primes, conditions, or modulates an excitability state of excitable biological tissue without directly evoking an action potential or other activation event.

“Depolarizing electric field” refers to an induced electric field sufficient to evoke, facilitate, initiate, or contribute to activation of excitable biological tissue.

“Modulating electrical activity” includes depolarization, hyperpolarization, inhibition, facilitation, synchronization, desynchronization, entrainment, conditioning, priming, recruitment, selective activation, alteration of excitability, alteration of propagation characteristics, or other alterations of bioelectrical or biophysical activity within excitable biological tissue.

“Closed-loop” refers to systems configured to adjust one or more stimulation parameters based on one or more sensed signals, including physiologic signals and/or signals representative of a magnetic field.

“Open-loop” refers to systems configured to deliver stimulation without feedback-based adjustment of stimulation parameters.

“Hybrid-loop” refers to systems configured to operate selectively or concurrently in open-loop and closed-loop modes.

“Neural state estimation” refers to the process of estimating, inferring, predicting, modeling, or otherwise determining a functional condition, state, or characteristic of excitable biological tissue based on one or more characteristics of one or more sensed signals, including physiologic signals and/or signals representative of a magnetic field. The functional condition may include one or more characteristics associated with neural activity, excitability, recruitment, propagation, synchrony, activation threshold, spatial distribution of activation, conduction properties, or other physiologic or biophysical characteristics. Estimation may be derived from observable signal characteristics, modeled characteristics, inferred characteristics, or combinations thereof, rather than direct measurement of the tissue state.

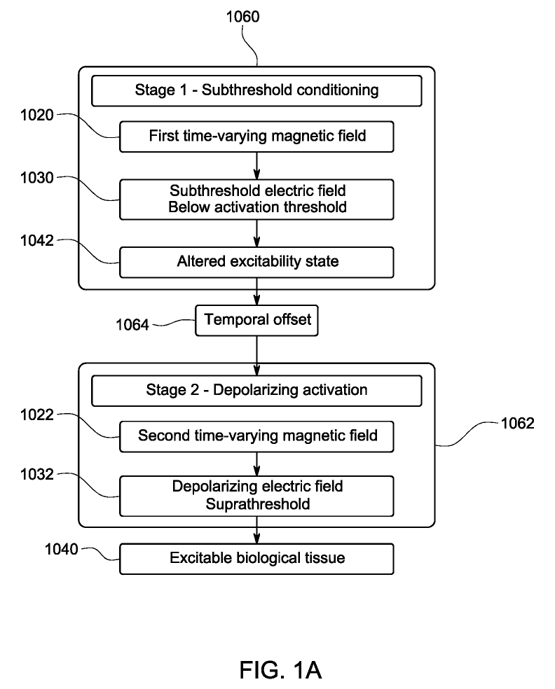

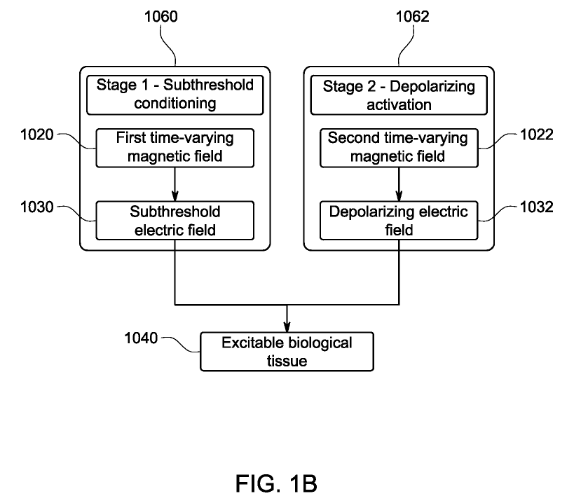

Referring to FIG. 1A, a two-stage modulation process with sequential delivery is shown. As in FIG. 1A, the process includes Stage 1: subthreshold conditioning 1060 and Stage 2: depolarizing activation 1062, both acting on excitable biological tissue 1040, which may influence an activation response within the tissue. Unlike FIG. 1B, the two stages in FIG. 1A are separated in time by a temporal offset 1064.

In Stage 1: subthreshold conditioning 1060, the first time-varying magnetic field 1020 induces the subthreshold electric field 1030 within the excitable biological tissue 1040. The subthreshold electric field 1030 is below the activation threshold and produces an altered excitability state 1042 in the excitable biological tissue 1040 without evoking an action potential. Following Stage 1: subthreshold conditioning 1060, the temporal offset 1064 separates the first stage from the second stage. The temporal offset 1064 represents a time interval between the cessation of the subthreshold electric field 1030 and the initiation of the depolarizing electric field 1032.

In Stage 2: depolarizing activation 1062, the second time-varying magnetic field 1022 induces the depolarizing electric field 1032 within the excitable biological tissue 1040. The depolarizing electric field 1032 is suprathreshold and sufficient to evoke activation.

Referring to FIG. 1B a two-stage modulation process for modulating electrical activity in excitable biological tissue with simultaneous delivery is shown. The two-stage modulation process includes a first stage, identified as Stage 1: subthreshold conditioning 1060, and a second stage, identified as Stage 2: depolarizing activation 1062. In the simultaneous delivery configuration shown in FIG. 1B, Stage 1 1060 and Stage 2 1062 operate concurrently, with both stages acting on the excitable biological tissue 1040 simultaneously.

In Stage 1: subthreshold conditioning 1060, a first time-varying magnetic field 1020 is generated that induces a subthreshold electric field 1030 within the excitable biological tissue 1040. The subthreshold electric field 1030 alters, biases, primes, conditions, or otherwise modulates an excitability state of the excitable biological tissue 1040 while simultaneously interacting with depolarizing activation associated with Stage 2 1062

In Stage 2: depolarizing activation 1062, a second time-varying magnetic field 1022 is generated that induces a depolarizing electric field 1032 within the excitable biological tissue 1040. The depolarizing electric field 1032 is sufficient to evoke activation in the excitable biological tissue 1040.

The combined effect of Stage 1: subthreshold conditioning 1060 and Stage 2: depolarizing activation 1062 on the excitable biological tissue 1040 may influence an activation response within the tissue. The subthreshold electric field 1030, which is the first induced electric field, establishes a subthreshold excitability state in the excitable biological tissue 1040 that lowers a depolarization threshold, thereby altering the response of the excitable biological tissue 1040 to the depolarizing electric field 1032, which is the second induced electric field. Together, the subthreshold electric field 1030 and the depolarizing electric field 1032 modulate electrical activity within the excitable biological tissue 1040 by influencing depolarization, hyperpolarization, inhibition, facilitation, synchronization, desynchronization, entrainment, subthreshold activity, propagation of electrical activity, excitability, recruitment characteristics, spatial distribution of activation, or combinations thereof, depending on one or more characteristics of the induced electric fields including amplitude, temporal profile, phase relationships, spatial orientation, spectral composition, or combinations thereof.

In one embodiment, the two-stage modulation shown in FIG. 1B is performed with a subthreshold electric field and a depolarizing electric field delivered simultaneously. In this embodiment, initial parameters of the first time-varying magnetic field 1020 and the second time-varying magnetic field 1022 may be predetermined, and the two-stage modulation process may operate in an open-loop, closed-loop, or hybrid configuration.

The first and second induced electric fields may have matching values for one or more parameters selected from frequency, amplitude, phase polarity, relative phase, or harmonic content.

In the sequential delivery configuration of FIG. 1A, the depolarizing electric field 1032 is generated after the subthreshold electric field 1030 has ceased, with the temporal offset 1064 separating the two stages. In the simultaneous delivery configuration of FIG. 1B, the subthreshold electric field 1030 is generated simultaneously with the depolarizing electric field 1032.

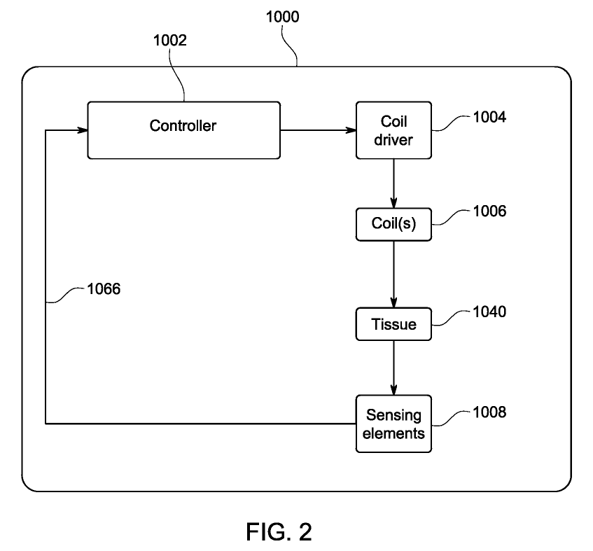

Referring to FIG. 2, a device 1000 for modulating electrical activity in excitable biological tissue 1040 is shown. The device 1000 includes a controller 1002, a coil driver 1004, a coil assembly 1006, and sensing elements 1008. Together, these components enable the device 1000 to generate one or more time-varying magnetic fields that induce electric fields within the excitable biological tissue 1040, to sense physiologic responses from the excitable biological tissue 1040, and to adjust stimulation parameters based on the sensed responses. In certain embodiments, the device 1000 may operate in an open-loop, closed-loop, and/or hybrid configuration.

The controller 1002 serves as a control system for the device 1000 and is configured to adjust one or more stimulation parameters including amplitude, timing, pulse width, phase, frequency, duty cycle, waveform characteristics, coil orientation, spatial positioning, phase relationships, spectral composition, or combinations thereof associated with one or more signals used to drive the coil assembly 1006. The controller 1002 is configured to provide one or more control signals to the coil driver 1004 based on the one or more stimulation parameters and may further be configured to maintain a target response, modulate electrical activity, estimate a state of the excitable biological tissue 1040, compensate for physiologic variability or changes in tissue conditions, or adjust one or more characteristics of one or more induced electric fields and/or corresponding physiologic responses.

In certain embodiments, the controller includes supervisory control logic configured to detect one or more conditions including unreliable sensing, excessive stimulation artifact, signal saturation, signal dropout, unexpected physiologic signal characteristics, excessive deviation from a target operating range, instability in control response, or other conditions associated with reduced reliability, performance, or safety of the control system. In response to such conditions, the controller may initiate a fallback mode comprising one or more of: transition to an open-loop or hybrid control mode, reduction or modification of one or more stimulation parameters, modification of one or more waveform characteristics, selection of one or more alternative sensing modalities, use of predefined stimulation parameters, limitation of operation to one or more predefined operating ranges, temporary suspension of stimulation, or combinations thereof. In some embodiments, the system records one or more sensed signals, stimulation parameters, control states, estimated tissue states, fallback events, or combinations thereof for subsequent analysis, clinician review, diagnostic evaluation, or adaptive optimization.

The coil driver 1004 receives one or more control signals from the controller 1002 and converts the one or more control signals into one or more drive signals for the coil assembly 1006, supplying electrical current to the coil assembly 1006 according to one or more parameters including amplitude, timing, pulse width, phase, frequency, duty cycle, waveform characteristics, coil orientation, spatial positioning, phase relationships, spectral composition, or combinations thereof specified by the controller 1002 to generate one or more time-varying magnetic fields that induce one or more electric fields within the excitable biological tissue 1040. The coil assembly 1006 includes one or more coils configured to generate one or more time-varying magnetic fields that induce one or more electric fields within the excitable biological tissue 1040. The one or more coils of the coil assembly 1006 may include planar coils, cylindrical coils, microcoils (µcoils), inductors, or combinations thereof.

Stimulation by the device 1000 is delivered without direct electrical contact between the coil assembly 1006 and the excitable biological tissue 1040, and does not require a direct electrode-tissue interface. One or more coils of the coil assembly 1006 generate one or more time-varying magnetic fields that induce one or more electric fields within the excitable biological tissue 1040. The absence of a direct electrode-tissue interface may reduce electrochemical interactions at a tissue surface, reduce susceptibility to impedance changes associated with fibrotic encapsulation, glial scar formation, or other biological barriers, and reduce risks associated with direct current injection into tissue. One or more induced electric fields may be generated across intervening media including blood, cerebrospinal fluid, connective tissue, encapsulation tissue, fibrotic regions, or combinations thereof, thereby enabling modulation of tissue regions that may be difficult to access using direct electrode contact.

In certain embodiments, the controller is further configured to compensate for changes in a stimulation environment associated with fibrotic encapsulation, scar tissue formation, glial response, connective tissue growth, cerebrospinal fluid variation, anatomical variability, positional changes, changes in spacing between the coil assembly and the excitable biological tissue 1040, or combinations thereof. Compensation may be achieved by adjusting one or more stimulation parameters associated with one or more time-varying magnetic fields and corresponding induced electric fields, including amplitude, waveform characteristics, phase, timing, frequency, spectral composition, spatial distribution, coil selection, coil orientation, phase relationships, or combinations thereof, based on one or more sensed physiologic responses, one or more sensed field characteristics, one or more estimated tissue states, or combinations thereof, to maintain one or more target induced electric field characteristics, activation states, recruitment characteristics, spatial distributions of activation, and/or corresponding physiologic responses despite variability in one or more intervening biological media.

The sensing elements 1008 are configured to detect one or more physiologic responses from the excitable biological tissue 1040, including one or more evoked responses. Sensed signals may include evoked compound action potentials (ECAPs), late responses including F-waves, H-reflexes, and A-waves, evoked synaptic activity potentials (eSAPs), electromyographic (EMG) signals, electrocardiographic signals, intracardiac electrograms, electrograms, field potentials, and other electrophysiological responses. Sensed parameters may further include conduction velocity, rheobase, chronaxie, recruitment characteristics, activation thresholds, propagation characteristics, synchrony characteristics, spatial distributions of activation, and other characteristics associated with responses of excitable biological tissue 1040.

Conventional electrode-based stimulation systems produce a stimulus artifact that may require a blanking interval, defined as a period during or after delivery of a stimulation pulse during which sensing is reduced, suppressed, filtered, or otherwise modified to avoid saturation or interference within sensing circuitry. Because one or more induced electric fields generated by the coil assembly 1006 arise from one or more time-varying magnetic fields rather than direct electrical contact with the excitable biological tissue 1040, a corresponding stimulus artifact may be reduced, redistributed, temporally separated, spatially differentiated, spectrally differentiated, or otherwise differ in amplitude, temporal profile, spatial characteristics, or frequency characteristics relative to electrode-based stimulation systems.

In some embodiments, differences in stimulus artifact associated with inductive stimulation permit reduction of a blanking interval and may enable the sensing elements 1008 to detect one or more evoked responses during or immediately following stimulation, including within an early post-stimulation time window in which the stimulus artifact does not fully obscure physiologic signals. Earlier detection of evoked responses may enable characterization of latency, onset timing, recruitment behavior, and early response components for real-time adjustment of one or more stimulation parameters, which may be more difficult or less reliable in systems requiring longer blanking intervals. In certain embodiments, detection and characterization of evoked responses are performed using one or more signal processing approaches including correlation-based methods, derivative-based methods, template matching, feature extraction, spectral analysis, adaptive filtering, or combinations thereof.

In some implementations, a correlation-based method is used to identify an evoked response based on similarity to a predefined, adaptive, or dynamically updated waveform template, while a derivative-based method is used to determine onset timing, latency, slope characteristics, inflection characteristics, peak characteristics, or combinations thereof.

In certain embodiments, outputs from multiple signal-processing methods are combined to improve detection reliability, reduce sensitivity to stimulation artifact, improve discrimination between physiologic and non-physiologic signals, and enhance robustness under conditions in which stimulation artifact exhibits extended temporal characteristics, overlapping responses, or complex morphology.

In certain embodiments, one or more signal-processing operations are implemented using one or more analog circuits, digital circuits, processors, programmable logic devices, computational elements, or combinations thereof, and are not limited to any specific algorithm, model, architecture, or implementation. In certain embodiments, the sensing elements 1008 are configured to detect one or more physiologic responses without application of a blanking interval, including during or immediately following delivery of one or more time-varying magnetic fields, provided that the physiologic response remains detectable in the presence of stimulation artifact.

In some implementations, differences in spatial, temporal, and/or spectral characteristics of stimulus artifact associated with inductive stimulation permit detection of one or more evoked responses in the presence of residual artifact, thereby enabling continuous, substantially continuous, intermittent, or periodic sensing under selected operating conditions. Evoked responses detected by the sensing elements 1008 may remain detectable at higher stimulation frequencies, including frequencies at which such responses are reduced, obscured, diminished, or not observable using conventional stimulation approaches. Detectability at higher stimulation frequencies may enable characterization of neural activity, monitoring of recruitment characteristics, neural state estimation, and adjustment of one or more stimulation parameters under operating conditions in which conventional systems may not provide reliable feedback.

A feedback path 1066 connects the sensing elements 1008 to the controller 1002, enabling the controller 1002 to receive one or more sensed physiologic responses and/or signals representative of one or more time-varying magnetic fields. Through the feedback path 1066, the controller 1002 adjusts one or more stimulation parameters associated with one or more time-varying magnetic fields and corresponding induced electric fields based on the sensed signals. In certain embodiments, the controller 1002 is configured to deliver one or more calibration, probing, conditioning, or test stimulation events to assess excitability, recruitment threshold, activation threshold, evoked response characteristics, field-response relationships, or combinations thereof. Information derived from such stimulation events may be used to initialize, update, optimize, recalibrate, or otherwise modify one or more control parameters including target operating ranges, gain parameters, response thresholds, stimulation thresholds, or combinations thereof.

In closed-loop embodiments, the device 1000 iteratively generates one or more time-varying magnetic fields, senses one or more physiologic responses via the sensing elements 1008, and adjusts one or more stimulation parameters associated with one or more time-varying magnetic fields and corresponding induced electric fields to modulate electrical activity of the excitable biological tissue 1040. Feedback derived from one or more sensed signals is used to maintain one or more target responses, dynamically adjust stimulation parameters, compensate for physiologic variability, and optimize energy delivery.

The controller 1002 is configured to adjust one or more stimulation parameters associated with one or more time-varying magnetic fields and corresponding induced electric fields, including amplitude, frequency, pulse width, waveform characteristics, temporal characteristics, phase relationships, spatial distribution, spectral composition, or combinations thereof, to produce one or more measurable physiologic responses within the excitable biological tissue 1040. Measurable physiologic responses may include evoked compound action potentials (ECAPs), electromyographic (EMG) signals, electrocardiographic signals, intracardiac electrograms, evoked synaptic activity potentials (eSAPs), field potentials, electrograms, or other electrophysiological responses. The controller 1002 may further adjust one or more stimulation parameters to maintain one or more evoked response characteristics within a predefined range, including one or more target response amplitudes, latencies, recruitment characteristics, or activation states associated with the excitable biological tissue 1040. Closed-loop control by the controller 1002 may further include neural state estimation based on one or more characteristics of one or more sensed physiologic signals received via the feedback path 1066, including signal presence, signal reduction, signal saturation, latency, waveform morphology, recruitment behavior, temporal patterns, spectral characteristics, synchrony characteristics, or combinations thereof. The controller 1002 adjusts one or more stimulation parameters associated with one or more time-varying magnetic fields and corresponding induced electric fields to navigate between functional states of neural activity. Neural state estimation does not require detection of an evoked response following every stimulation event. Instead, a neural state may be inferred from trends, temporal changes, or variations in signal characteristics observed across detected responses, including changes in amplitude, latency, morphology, synchrony, or recruitment behavior over time. The sensing elements 1008 may operate continuously, substantially continuously, intermittently, periodically, adaptively, or combinations thereof depending on the application.

Electrical and magnetically induced signals detected by the sensing elements 1008 may be combined to form hybrid evoked activity potentials (HEAPs), which provide additional sensing information for feedback-based control, neural state estimation, adaptive modulation, or combinations thereof by the controller 1002.The controller 1002 is further configured to control one or more spatial characteristics of one or more induced electric fields by adjusting one or more parameters associated with one or more time-varying magnetic fields, including coil orientation, phase relationships, waveform characteristics, spectral composition, spatial positioning, timing, or combinations thereof, to control one or more properties of the induced electric fields within the excitable biological tissue 1040.

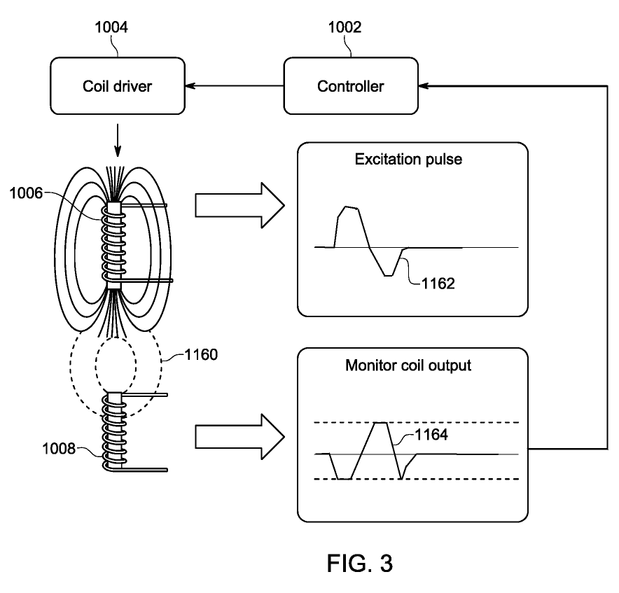

Referring to FIG. 3, a coil assembly 1006 and one or more sensing elements 1008 are shown in relation to a controller 1002 and a coil driver 1004. The controller 1002 provides one or more excitation signals 1162 to the coil driver 1004. The coil driver 1004 drives the coil assembly 1006 to generate one or more time-varying magnetic fields 1160. The one or more time-varying magnetic fields 1160 propagate from the coil assembly 1006 into surrounding tissue and induce one or more electric fields within the excitable biological tissue 1040. The sensing elements 1008 are positioned to detect one or more signals representative of the magnetic field 1160 generated by the coil assembly 1006. One or more sensed signals 1164 are returned to the controller 1002, enabling the controller 1002 to characterize one or more properties of the magnetic field 1160 and/or corresponding induced electric fields produced by the coil assembly 1006. The excitation signals 1162 and sensed signals 1164 are illustrated as representative waveforms associated with operation of the controller 1002, coil driver 1004, coil assembly 1006, and sensing elements 1008.

Referring to FIG. 3, one or more coils are configured to generate the first time-varying magnetic field, and one or more additional coils, positioned proximal, distal, adjacent, or otherwise spatially offset relative to at least one of the one or more coils, are configured to operate in a passive sensing mode to detect one or more signals representative of the first time-varying magnetic field generated by the one or more coils.

One or more detected signals are used to estimate one or more characteristics of the generated magnetic field, one or more corresponding induced electric fields, spatial distributions thereof, or combinations thereof, and to adjust one or more stimulation parameters associated with the one or more time-varying magnetic fields and corresponding induced electric fields. In certain embodiments, spatial orientation, spatial distribution, directional characteristics, gradients, and/or interference characteristics of one or more induced electric fields are controlled to selectively modulate one or more regions, pathways, or components of the excitable biological tissue 1040.

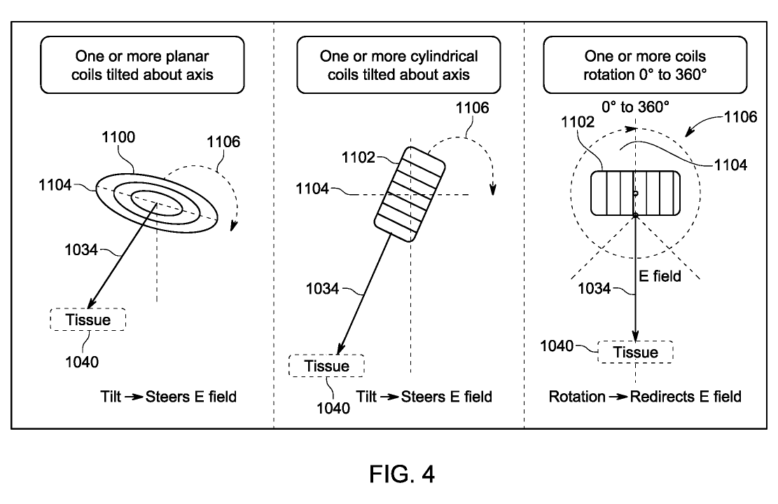

The coil(s) 1006 (see FIG. 2) may include two or more coils oriented using one or more configurations shown in FIG. 4 such that corresponding induced electric fields interact within the excitable biological tissue 1040. Such interaction may include constructive interference resulting in increased effective field strength within one or more target regions, destructive interference resulting in reduced field strength within one or more non-target regions, or combinations thereof. Interference characteristics may be controlled by adjusting one or more parameters including relative phase, timing, amplitude, waveform characteristics, spectral composition, spatial positioning, orientation, or combinations thereof, to enhance modulation within one or more first regions of the excitable biological tissue 1040 while reducing modulation within one or more second regions.

In the left panel of FIG. 4, a planar coil 1100 is positioned relative to excitable biological tissue 1040 and is configured to tilt about one or more tilt axes 1104 at one or more tilt angles 1106. Tilting of the planar coil 1100 about the one or more tilt axes 1104 steers one or more induced electric field vectors 1034 relative to the excitable biological tissue 1040. By adjusting the one or more tilt angles 1106, spatial orientation of the induced electric field vectors 1034 is altered, enabling one or more induced electric fields to be directed toward one or more target regions of the excitable biological tissue 1040.

In the center panel of FIG. 4, a steering configuration using one or more cylindrical coils is shown. A cylindrical coil 1102 is positioned relative to excitable biological tissue 1040 and is configured to tilt about one or more tilt axes 1104 at one or more tilt angles 1106. Tilting of the cylindrical coil 1102 about the one or more tilt axes 1104 steers one or more induced electric field vectors 1034 relative to the excitable biological tissue 1040. The cylindrical coil 1102 provides an alternative coil geometry for generating and steering one or more time-varying magnetic fields and corresponding induced electric fields relative to the planar coil 1100.

In the right panel of FIG. 4, a steering configuration using one or more rotational coil arrangements is shown. A cylindrical coil 1102 is positioned relative to excitable biological tissue 1040 and is configured to rotate about one or more rotational axes 1104 over one or more rotational paths extending through a range up to 360 degrees. Rotation of the cylindrical coil 1102 redirects one or more induced electric field vectors 1034 relative to the excitable biological tissue 1040. One or more tilt angles 1106 are shown relative to the one or more rotational axes 1104. The combination of tilt and rotation enables one or more induced electric field vectors 1034 to be steered in a plurality of directions relative to the excitable biological tissue 1040. The steering configurations of FIG. 4 enable one or more time-varying magnetic fields and corresponding induced electric fields to be oriented such that one or more induced electric field vectors align with one or more structural orientations of the excitable biological tissue 1040.

In certain embodiments, structural orientation includes one or more directional, anisotropic, conductive, or functional characteristics of the excitable biological tissue 1040. Where the excitable biological tissue 1040 comprises neural tissue, one or more induced electric field vectors 1034 may be steered to align with one or more axonal directions to influence modulation of electrical activity. Where the excitable biological tissue 1040 comprises cardiac tissue, one or more induced electric field vectors 1034 may be steered to align with myocardial fiber orientations to influence conduction pathways, activation characteristics, or combinations thereof. Steering comprises controlling one or more spatial orientations of one or more induced electric fields relative to the excitable biological tissue 1040.

Spatial gradients of one or more induced electric fields may be configured to differentially modulate one or more regions, pathways, structures, or components within the excitable biological tissue 1040. A spatial gradient of an induced electric field corresponds to a spatial rate of change of one or more characteristics of the induced electric field across the excitable biological tissue 1040. By adjusting one or more coil orientations, positions, geometries, phase relationships, or combinations thereof, one or more spatial gradients may be tailored to preferentially influence selected regions, pathways, or cell populations within the excitable biological tissue 1040 while reducing effects in other regions. Such gradients may produce differential polarization along one or more cellular structures including neural fibers, myocardial fibers, cellular networks, or combinations thereof, resulting in selective activation, inhibition, facilitation, conditioning, or modulation of different regions or pathways. One or more induced electric field vectors 1034 may be configured, through control of spatial distribution, orientation, gradients, or combinations thereof, to preferentially modulate selected regions of the excitable biological tissue 1040 while reducing off-target activation. Resulting activation may include modulation of central neural structures, peripheral neural structures, cardiac structures, autonomic structures, sensory fibers, motor fibers, autonomic fibers, cutaneous receptors, or combinations thereof.

The methods and devices described herein are applicable to any excitable biological tissue 1040 including neural tissue, cardiac tissue, smooth muscle tissue, skeletal muscle tissue, autonomic tissue, enteric tissue, sensory tissue, or other tissues comprising electrically responsive cells or cellular networks. One or more parameters associated with the first time-varying magnetic field 1020 and the second time-varying magnetic field 1022, including amplitude, frequency, pulse width, timing, waveform characteristics, phase relationships, spatial distribution, spectral composition, or combinations thereof, may be selected based on the target excitable biological tissue 1040 and one or more desired modulation outcomes. While presently preferred embodiments have been described, those of ordinary skill in the art will recognize that modifications, combinations, substitutions, and variations are possible within the scope of the appended claims. Such variations may include alternative waveform combinations, alternative coil geometries, additional sensing modalities, alternative control architectures, combinations of open-loop, closed-loop, and hybrid control, alternative signal-processing approaches, alternative state-estimation methods, or combinations thereof. The described embodiments are illustrative and are not intended to limit the scope of the appended claims.

What is claimed is:

1. A method of modulating electrical activity in excitable biological tissue using an induced electric field generated by a time-varying magnetic field, the method comprising:
generating a first time-varying magnetic field configured to induce a subthreshold electric field within the excitable biological tissue, the subthreshold electric field altering an excitability state of the tissue without evoking an action potential; and
generating a second time-varying magnetic field configured to induce a depolarizing electric field within the excitable biological tissue sufficient to evoke activation;
wherein a first induced electric field establishes a subthreshold excitability state in the excitable biological tissue that lowers a depolarization threshold, thereby altering a response of the excitable biological tissue to a second induced electric field.

2. The method of claim 1, wherein the first time-varying magnetic field and the second time-varying magnetic field have matching values for at least one parameter selected from frequency, amplitude, phase polarity, relative phase, or harmonic content, and the second induced electric field is generated simultaneously with the first induced electric field or after the first induced electric field has ceased.

3. The method of claim 1, wherein modulation is performed without sensing or adjusting based on a physiological response from the excitable biological tissue.

4. The method of claim 1, wherein one or more time-varying magnetic fields comprise waveforms having asymmetric or unbalanced characteristics, including one or more of unequal positive and negative phases, unequal temporal characteristics, unequal amplitude characteristics, unequal phase durations, unequal spectral characteristics, or combinations thereof, thereby generating one or more corresponding induced electric fields.

5. The method of claim 1, wherein the first or second induced electric fields are generated by one or more time-varying magnetic fields produced by one or more coils comprising planar coils, cylindrical coils, microcoils (µcoils), inductors, or combinations thereof.

6. The method of claim 1, wherein waveform characteristics are configured to influence electrical properties of the excitable biological tissue, including impedance or conductivity, thereby modifying responsiveness to stimulation.

7. The method of claim 1, wherein a single time-varying magnetic field waveform is configured to induce an electric field that produces both subthreshold modulation and depolarizing activation within the excitable biological tissue.

8. The method of claim 1, wherein two or more time-varying magnetic fields generate corresponding induced electric fields that interact within the excitable biological tissue to produce constructive interference that modifies one or more characteristics of an effective induced electric field within one or more regions of the excitable biological tissue to modulate activation thereof.

9. The method of claim 1, wherein one or more time-varying magnetic fields generate one or more induced electric fields having spatial gradients configured to differentially modulate one or more regions or components within the excitable biological tissue.

10. The method of claim 1, wherein the time-varying magnetic field is oriented such that induced electric field vectors are aligned with a structural orientation of the excitable biological tissue.

11. A method of modulating electrical activity in excitable biological tissue using an induced electric field generated by one or more time-varying magnetic fields, the method comprising:
generating a first time-varying magnetic field configured to induce a subthreshold electric field within the excitable biological tissue, the subthreshold electric field altering an excitability state of the tissue without evoking an action potential;
generating a second time-varying magnetic field configured to induce a depolarizing electric field within the excitable biological tissue sufficient to evoke activation;
sensing an evoked response from the excitable biological tissue;
adjusting one or more parameters associated with at least one of the first or second time-varying magnetic fields and corresponding induced electric fields based on the sensed evoked response, the one or more parameters including one or more of amplitude, phase, pulse width, frequency, waveform shape, or temporal characteristics, wherein one or more waveform characteristics, including temporal profile or frequency content, are selected to influence or control the induced electric field, a spatial distribution thereof, or a corresponding physiologic response; and
iteratively repeating the generating, generating, sensing, and adjusting steps in a closed-loop manner to modulate electrical activity of the excitable biological tissue by controlling one or more induced electric fields and one or more corresponding physiologic responses, based on the sensed evoked responses.

12. The method of claim 11, wherein one or more parameters associated with at least one of the first or second time-varying magnetic fields and corresponding induced electric fields are controlled, the one or more parameters including one or more of amplitude, phase, frequency, pulse width, waveform shape, temporal characteristics, and spatial distribution, such that one or more characteristics of the induced electric fields are controlled to produce a measurable physiologic response within the excitable biological tissue, the measurable physiologic response including one or more electrophysiological signals resulting from the induced electric fields.

13. The method of claim 11, wherein one or more parameters associated with the one or more time-varying magnetic fields and corresponding induced electric fields are adjusted to maintain one or more evoked response characteristics within a predefined range.

14. The method of claim 11, wherein adjusting is based on one or more response characteristics associated with excitability or activation of the excitable biological tissue.

15. The method of claim 11, wherein one or more coils are configured to deliver the first time-varying magnetic field, and one or more additional coils, positioned proximal, distal, or otherwise spatially offset relative to at least one of the coils configured to deliver the first time-varying magnetic field, are configured to operate in a passive sensing mode to detect a signal representative of the first time-varying magnetic field generated by the one or more coils, and wherein the detected signal is used to estimate one or more characteristics of the delivered magnetic field or a corresponding induced electric field and to adjust one or more stimulation parameters.

16. The method of claim 11, wherein sensed physiologic responses and/or signals associated with the one or more time-varying magnetic fields are processed to estimate one or more characteristics of a state of the excitable biological tissue, including excitability, recruitment, activation threshold, conduction properties, spatial distribution of activation, synchrony characteristics, or combinations thereof, and wherein one or more stimulation parameters associated with the time-varying magnetic fields and corresponding induced electric fields are adjusted based on the estimated state.

17. A device for modulating electrical activity in excitable biological tissue using one or more induced electric fields generated by one or more time-varying magnetic fields, the device comprising:
one or more coils configured to generate the one or more time-varying magnetic fields that induce the one or more electric fields within the excitable biological tissue; and
a control system configured to drive the one or more coils to produce a first time-varying magnetic field and a second time-varying magnetic field,
wherein the first and second time-varying magnetic fields are configured to induce respective electric fields that alter an excitability state of the excitable biological tissue and modulate electrical activity thereof.

18. The device of claim 17, further comprising a sensing system configured to detect one or more physiologic responses, including evoked responses, from the excitable biological tissue, and wherein the control system is configured to adjust one or more parameters associated with the one or more time-varying magnetic fields and corresponding induced electric fields based on the detected one or more physiologic responses.

19. The device of claim 17, wherein the device is configured to control one or more spatial characteristics of one or more induced electric fields by adjusting coil orientation, phase relationships, or waveform characteristics of one or more time-varying magnetic fields.

20. The device of claim 17, wherein the one or more coils comprise:
one or more planar coils configured to tilt about one or more axes to steer one or more induced electric fields; and
one or more cylindrical coils configured to tilt about one or more axes and/or to rotate about an axis over a range up to 360 degrees to steer one or more induced electric fields,
wherein steering comprises controlling spatial orientation of the one or more induced electric fields relative to the excitable biological tissue.
